(19) United States
(12) Patent Application Publication
(10) Pub. No.: US 2017/0191016 A1
(43) Pub. Date: Jul. 6, 2017

(54) ADJUSTABLE HEIGHT HARVEST VALVE ASSEMBLY FOR BIOREACTORS

(71) Applicant: PBS Biotech, Inc., Camarillo, CA (US)

(72) Inventors: Brian Lee, Thousand Oaks, CA (US); Gary Evans, Ventura, CA (US); Tom Zevin, Valencia, CA (US); Daniel Giroux, Cardiff by the Sea, CA (US); Yas Hashimura, Woodland Hills, CA (US); Oscar Garza, Ventura, CA (US)

(21) Appl. No.: 15/394,282

(22) Filed: Dec. 29, 2016

Related U.S. Application Data
(60) Provisional application No. 62/273,834, filed on Dec. 31, 2015.

(51) Int. Cl. C12M 1/00

(52) U.S. Cl. CPC C12M 23/50; C12M 23/28; C12M 23/14; C12M 23/26

(57) ABSTRACT

An adjustable harvest valve assembly for a bioreactor system which offers all of the benefits of a harvest port, while allowing cell-free liquid to be collected at various liquid heights and multiple times throughout the run.

### RELATED APPLICATION INFORMATION

This patent claims priority from the following provisional patent applications: Provisional Patent Application No. 62/273,834, entitled ADJUSTABLE HEIGHT HARVEST VALVE ASSEMBLY FOR BIOREACTORS, filed Dec. 31, 2015.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### FIELD OF THE INVENTION

An adjustable harvest valve assembly for a bioreactor system and methods of use therefore.

### BACKGROUND

Efforts of biopharmaceutical companies to discover new biological drugs have increased exponentially during the past two decades. Bioreactors have been used for cultivation of microbial organisms for production of various biological or chemical products in the pharmaceutical, biotechnological, and beverage industry. Most biological drugs are produced by cell culture or microbial fermentation processes which require sterile bioreactors and an aseptic culture environment.

A production bioreactor contains culture medium in a sterile environment that provides various nutrients required to support growth of the biological agents of interest. Stainless steel tanks with horizontal stirring mechanisms have long been the only option for large scale production of biological products in suspension culture. Manufacturing facilities with conventional stainless bioreactors, however, face numerous problems such as large capital investments for construction, high maintenance costs, long lead times, and inflexibilities for changes in manufacturing schedules and production capacities. Such bioreactors can only be reused for the next batch of biological agents after cleaning and sterilization of the vessel. These procedures require a significant amount of time and resources, especially to monitor and to validate each cleaning step prior to reuse for production of biopharmaceutical products.

Scaling up cell culture processes in bioreactors can pose numerous engineering challenges, much of which has been addressed and resolved for the therapeutic protein market, for which well-established cell lines such as CHO (Chinese hamster ovarian) are used. These cell lines, which have been adapted from adherent culture over time to grow in single-cell suspension, are fairly robust to shear stress and are even able to handle perfusion culture modes, where fresh cell culture medium is added to the bioreactor in a continuous manner as spent medium is withdrawn and cells are retained in the bioreactor.

Cells used in cell therapy market, however, are often primary cells such as mesenchymal stem cells and embryonic stem cells, newly derived from human donors and therefore are more shear-sensitive and are adherent-based in nature (i.e. they will grow in aggregates or on scaffolds such as microcarriers).

Cell culture medium exchanges for these primary cells are typically performed in discrete mode of removing spent medium first and then replacing with fresh medium, not in perfusion mode of removing and replacing medium continuously to avoid unnecessary shearing effect on cells. To remove spent medium from a bioreactor, agitation is first turned off and cells (in aggregates or on microcarriers) are allowed to settle to the bottom, before spent medium is removed.

In addition to medium exchanges, the method of removing and replacing cell-free liquid is also necessary during in situ harvest in a bioreactor, where spent medium is first withdrawn and then cells are rinsed using buffered saline solution in a number of wash steps before they are dissociated via enzyme and then quenched with medium. The in situ harvest process therefore adds to the number of cell-free liquid removal and addition steps required during a cell culture run, the total number for which could be up to 20 times per run.

Despite a proliferation of bioreactor designs for culturing primary cells, the options for cell culture medium exchange are relatively limited and time-consuming, and thus there is a need for a faster and easier technique.

### SUMMARY OF THE INVENTION

The present application discloses adjustable harvest valve assemblies for a bioreactor system which enables exchange/replenishment of cell culture medium in bioreactors used to culture primary cells.

### DESCRIPTION OF THE DRAWINGS

FIG. 1 an embodiment of the adjustable height harvest valve in the retracted position;

FIG. 2 shows the adjustable height harvest valve, in a fully extended position;

FIG. 3 illustrates the adjustable height harvest valve in a retracted position, with an optional locking mechanism to prevent radial and axial movement of the valve once position has been fixed;

FIG. 4A shows the adjustable height harvest valve in a fully extended position, with the optional locking mechanism keeping the valve position locked;

FIG. 4B is an enlarged view of the rotating flow gate valve mechanism; and

FIG. 5 is a perspective view of a small-volume bioreactor in which an adjustable height harvest valve of the present application can be utilized.

### DETAILED DESCRIPTION

The present application provides an adjustable harvest valve assembly for a bioreactor system which offers all of the benefits of a harvest port, while allowing cell-free liquid to be collected at various liquid heights and multiple times throughout the run, all of which are critical requirements when dealing with culturing of primary cells.

Current techniques are limited. In a small bench-top scale system such as a spinner flask, spent medium or other cell-free liquid is manually removed through a port with a removable cap using a pipette in a biosafety cabinet; for larger bioreactor systems, a harvest port or a dip tube would be required. Although the pipetting step at small scale is relatively straightforward, removing cell-free liquid out of a large bioreactor can be problematic for a number of reasons, and the present application contemplates a number of solutions, as follows:
1) A harvest port may be positioned at the bottom of the bioreactor for ease of liquid removal, but an open-tube configuration would allow cells to settle in the port opening during a run, which not only reduces the amount of cells that can be produced but also results in the plugging of the port. A plunger mechanism on the harvest port for opening the seal for harvesting could be added, but making it re-sealable as a liquid drain valve for multiple uses could be challenging.
2) A harvest port can be designed with an extension tall enough to allow cell-free liquid to be removed out of the bioreactor after cell settling, but the height would be fixed and would not allow the flexibility to withdraw liquid at different levels for various medium exchange and harvest volumes.
3) A dip tube has the advantage of allowing cell-free liquid to be skimmed from above the settled cells, but it must extend a relatively long distance since it is inserted from the top. At large scale (15 L and larger), this design can become rather unwieldy and impractical. Inserting a tube from the top also poses greater likelihood of hydrodynamic pinch points with other components in the bioreactor such as the impeller and sensors, which could result in unknown mechanical shear effects.
4) Shear effects need to be minimized for primary cell culture processes, as shear protectant such as Pluronic F-68 is not added to such cell culture mediums due to regulatory concerns. In the cell therapy application, cells are the final product to be manufactured, so adding any component that could affect the identity, viability, and potency of the cells is generally to be avoided.

In view of these challenges, an adjustable height harvest valve assembly is described which is mounted to and extends through the wall of a lower section of the bioreactor for ease of liquid removal. The assembly features a gate valve at the upper end of a hollow harvest tube that is mounted to the bottom wall and can be elevated into the bioreactor from below. The harvest tube slides through a harvest port in the bottom wall which provides a fluid seal therearound. In this way, the gate valve may be axially positioned at a desired height within the bioreactor for liquid removal.

In a preferred embodiment, the bioreactor includes a rigid outer container or housing (not shown) that receives a single-use bioreactor vessel 20 of sufficient size to contain a fluid to be mixed. A variety of different sizes of bioreactors are used from the maximum working volume of 3 L up to 500 L, and which can process various liquid volumes in each vessel. The bioreactor vessel 20 is preferably a disposable bioreactor bag 20 usually made of a three-layer plastic foil, such as polyethylene terephthalate, although the harvesting assembly described herein may also be used with a rigid bioreactor vessel.

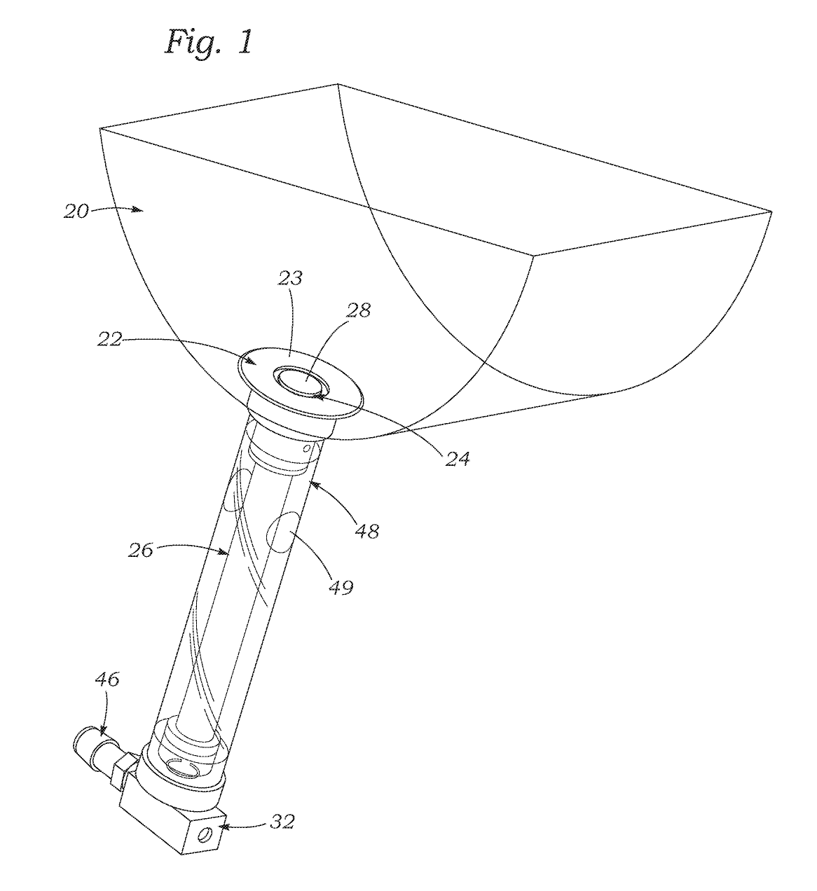

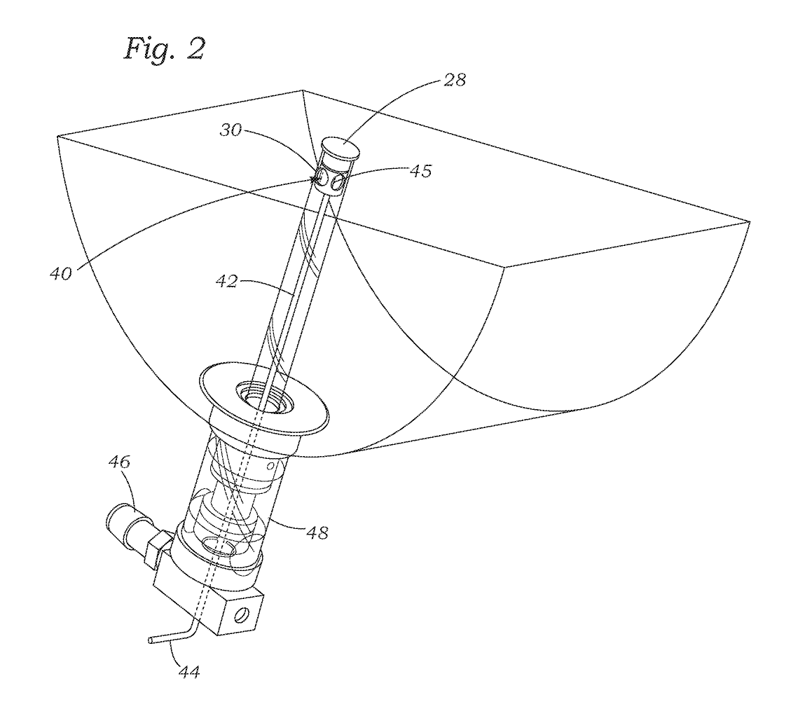

One embodiment of an adjustable height harvest valve assembly of the present application seen in FIGS. 1-2 consists of a harvest port 22 having a port disc flange 23 that is heat sealed or otherwise adhered to a bottom wall of the bioreactor bag 20. The port 22 defines a throughbore and desirably has circular grooves (not shown) internally spaced along its length to receive O-rings 24 therein for creating a dynamic liquid seal against a rigid tube 26 that can be moved axially within the throughbore to a desired height for liquid removal. The tube 26 has a cap 28 at a terminal end to prevent liquid flow when the tube is retracted into the port 22, as in FIG. 1, but hole(s) 30 in a side wall of the tube 26 spaced from the cap (see FIG. 2) to allow liquid flow into an interior of the tube 26 when extended. The hole(s) 30 form part of a gate valve 40 described below with reference to FIG. 2.

The rigid tube 26 is secured and mates with a lower rigid block 32 having a cross bore (not numbered), and a lower or outer aperture (not shown) of the tube 26 is positioned to align with the bore of the block 32.

A rotating flow gate valve 40 as seen in FIG. 2 may be secured within the rigid tube 26 and sealed to prevent fluid leak when it is rotated. In one embodiment, the gate valve 40 connects via a slender rod 42 within the tube 26 to a rotational actuating mechanism such as a manual stopcock lever 44 below the rigid block 32. The valve 40 also has aperture(s) 45 at the same axial position as the rigid tube 26 but offset by 90 degrees (¼ turn) or other predetermined amount to allow liquid to flow through the rigid tube 26 and bore in the rigid block 32 only after the valve has been rotated accordingly to align the holes 30 with the apertures 45.

The block 32 further mates and seals with a tubing connector 46, which has a hose barb or other coupler for attaching silicone tubing or other tubing for biopharmaceutical use as desired by the end user. If desired, rigid block 32 and tubing connector 46 could be manufactured as one part, but in Error! Reference source not found. two parts are shown, as tubing connector 46 is an off-the-shelf component that is readily available in the market and rigid block 32 is more easily machined by itself.

A longitudinally flexible sheath 48, secured around the valve assembly maintains sterility of inner components during valve movements. As seen in FIG. 1, the flexible sheath 48 is extended, while in FIG. 2 the sheath 48 is longitudinally collapsed. The sheath 48 may contain one or more vents 49 such as patches with sterilizing-grade (0.2-micron pore size or smaller) and gamma radiation stable membrane such as Tyvek® to alleviate positive or negative pressure generation in the section between the sheath 48 and tube 26 during valve movement. The sheath 48 can also be made entirely out of this gas permeable material, instead of utilizing patches.

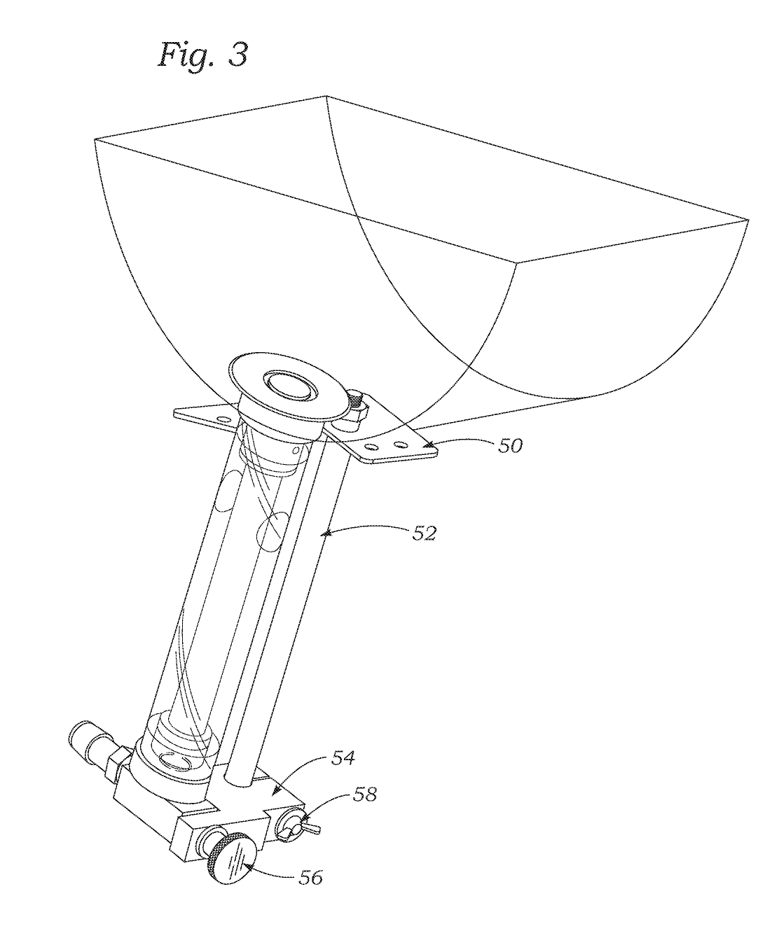

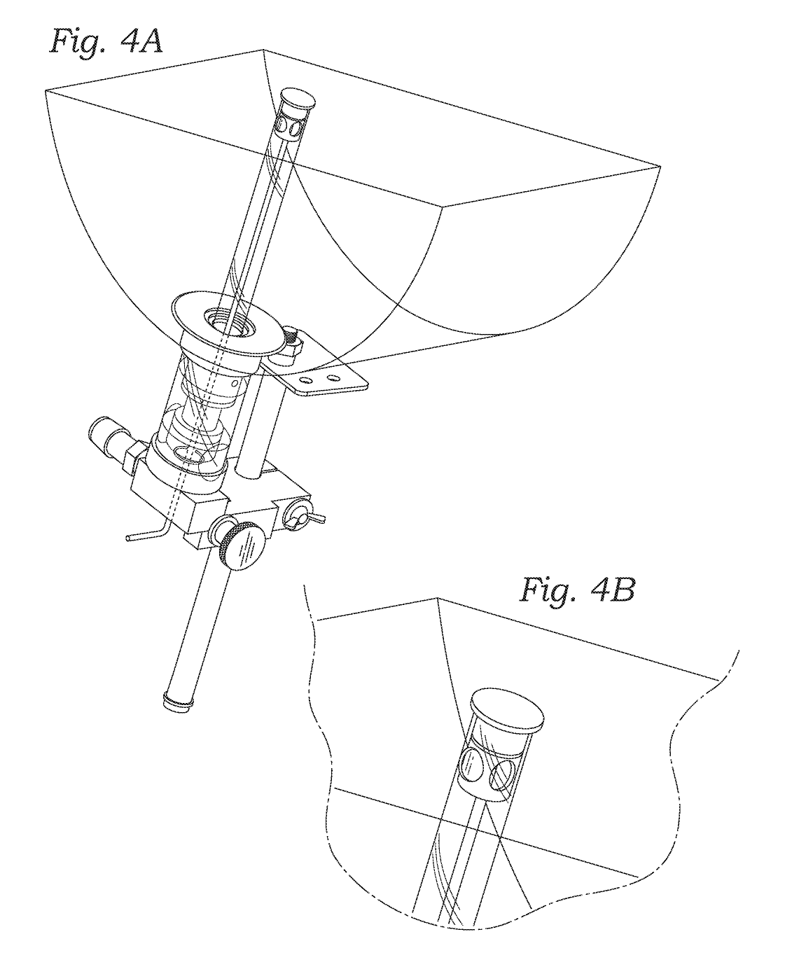

An optional locking mechanism may also be used with the adjustable harvest valve assembly to maintain the position of the valve. One embodiment of this locking mechanism shown in FIGS. 3, 4A and 4B consists of a mounting plate 50 for securing it onto the outer rigid bioreactor housing (again, not shown), secured to a rigid rod 52 that extends down about the same length as the harvest tube 26. A rigid valve positioning block 54 may slide along the length of the rigid rod 52 through a bifurcated section thereof. The positioning block 54 is secured against the rigid block 32 on the valve assembly with a screw 56, for example. Once the valve has been set to the desired height, a clamping screw 58 tightens the two parts of the bifurcated section of the positioning block 54 to lock in the axial position of the valve.

The adjustable height harvest valve assembly is desirably manufactured, packaged, and shipped to the user in the position as depicted in Error! Reference source not found., so that at the start of a cell culture run, the port 22 remains plugged. There could be a mechanism on the bag assembly to ensure the valve is not inadvertently extended, either during shipping or by the user during bag installation into the bioreactor housing, such as for instance the locking mechanism described above.

During medium exchange/harvest step, and once impeller agitation is stopped and cells are allowed to settle to the bottom of the bioreactor bag 20, the user manually extends the rigid tube 26 to an extended position within the bag. Even though Figure shows the fully extended position, tube 26 may be extended to any height between the maximum and minimum extension (while still exposing the gate valve 40 to fluid). The valve 40 is then turned 90 degrees (¼ turn) or other predetermined amount about its axis to align the hole(s) 30 on rigid tube 26 and apertures 45 on the rotating flow gate valve 40 and allow liquid to flow through the rigid tube 26, rigid block 32, and tubing connector 46 with flexible biopharmaceutical tubing. To stop the flow of liquid, the user rotates the valve 40 about its axis back to the position as depicted in Figure. The valve assembly is then retracted fully to plug the port 22, as depicted in Error! Reference source not found.. A similar sequence is shown in FIGS. 3, 4A and 4B for the embodiment having the mechanism for locking the position of the valve assembly relative to the bioreactor vessel.

The adjustable height harvest valve assembly is shown mounted to a bottom wall of the bioreactor bag 20, which is desirable as it facilitates fluid flow by gravity. However, the valve assembly may be positioned at various locations around the bioreactor bag 20, and fluid extraction may be activated with suction.

In another embodiment of the adjustable height harvest valve assembly, there is no rotating valve 40 for aligning holes(s) with the outer tube 26, and cell-free liquid is allowed to flow out once the rigid tube 26 is extended into the bioreactor bag. A clamp, stopcock, or other such valve is then loosened or removed on the flexible tubing attached to the tubing connector 46 to initiate flow. In this version of the design, there would be no rotating movement of the valve 40 but only an axial movement of the tube 26. This embodiment offers the advantage of having a simpler design for manufacturing and operation. However, the no valve option could allow some cell collection in the rigid tube 26 in the first instance of using this valve assembly for each run as there would be no liquid in the tube 26. The valve prevents any ingress of fluid prior to elevating the tube 26 to the desired height.

The adjustable harvest valve assembly as depicted in Error! Reference source not found. and Figure would most likely be a part of the single-use bag assembly and would be packaged and sterilized by gamma radiation, to be used in a single-use bioreactor system, but this design could also be applied to conventional stainless steel bioreactor systems as well. The optional locking mechanism as depicted in FIG. 2, Figure A and 4B would be a reusable component of the bioreactor housing assembly, as part of either a single-use bioreactor system or a conventional stainless steel bioreactor system.

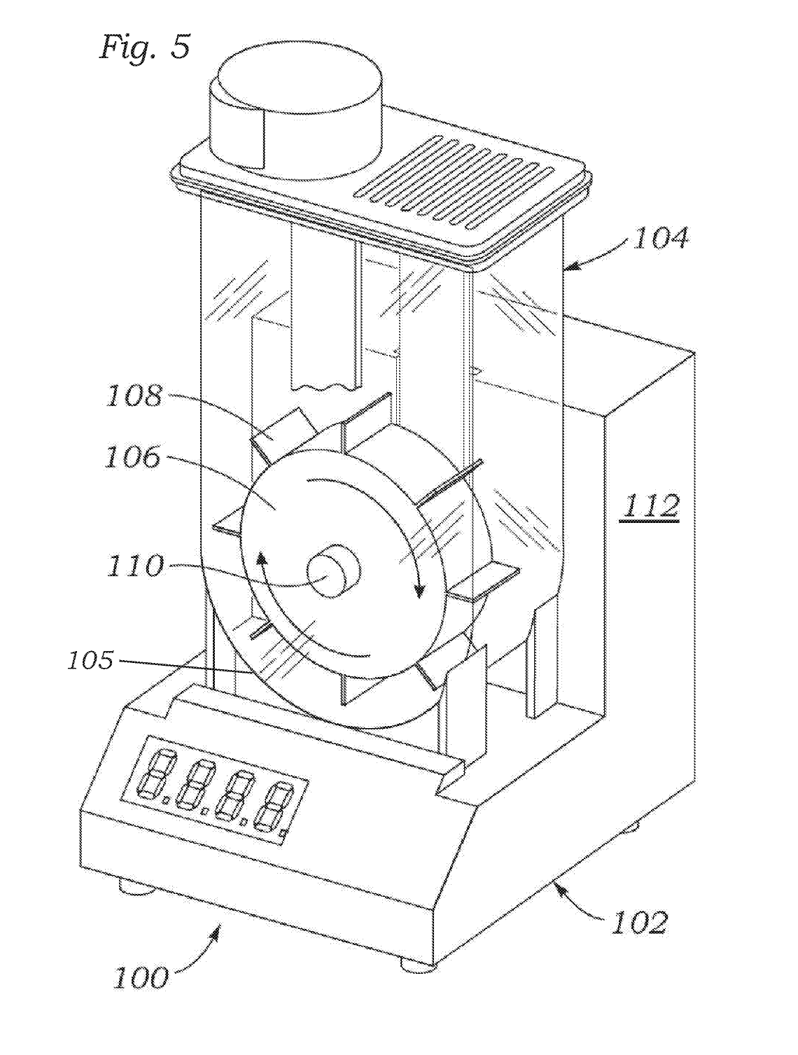

FIG. 5 illustrates an exemplary embodiment of a small-volume bioreactor 100 in which the adjustable height harvest valve described herein can be utilized. The bioreactor 100 comprises a base unit 102 supporting a disposable container 104. The container 104 preferably has a generally rectangular upper section and a semi-cylindrical lower section 105, as shown. The container 104 is preferably a single-use disposable bag which may be supported within a rigid outer housing of the same shape. The aforementioned adjustable harvest valve assembly is desirably mounted to and extends through the wall of the semi-cylindrical lower section 105.

A mixing or agitating wheel 106 is mounted wholly within the container 104 for rotation within the semi-cylindrical lower section. Preferably, the wheel 106 features a series of vanes 108 on its exterior for stirring the solution within the container 104, and also preferably includes inner vanes (not shown). The wheel 106 rotates about a horizontal axis on hubs 110 secured to the front and/or back walls of the container 104 (i.e., only one wheel hub 110 may be secured to the container 104). In a preferred embodiment, the base unit 102 includes an upstanding cabinet 112 within which is housed a drive system including rotating magnets (not shown). Corresponding magnets or ferromagnetic material mounted around the wheel 106 allow coupling of the drive system to enable rotation of the wheel from outside the container 104, thus eliminating seals and the like which might contaminate the solution within the container. In a preferred embodiment, the volume capacity of the container 104 is between 0.05-1.0 L, although the system can be scaled up for larger capacities.

The illustrated bioreactor 100 is for use inside CO₂ incubators, which are typically run with temperature control and with a fixed percentage of CO₂ in air. Consequently, independent pH and DO controls for the bioreactor 100 are not necessary.

### Closing Comments

Throughout this description, the embodiments and examples shown should be considered as exemplars, rather than limitations on the apparatus and procedures disclosed or claimed. Although many of the examples presented herein involve specific combinations of method acts or system elements, it should be understood that those acts and those elements may be combined in other ways to accomplish the same objectives. Acts, elements and features discussed only in connection with one embodiment are not intended to be excluded from a similar role in other embodiments.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

It is claimed:

1. A bioreactor harvest valve assembly, comprising:
a. a harvest port adapted to be mounted in and through a wall of a bioreactor vessel and having a throughbore;
b. a harvest tube configured to pass through the throughbore of the harvest port which has seals therein to prevent leakage around the harvest tube, the harvest tube having a cap at a terminal end that seals against the harvest port and closes the throughbore in a retracted position of the harvest tube;
c. one or more holes formed in a side wall of the harvest tube spaced from the cap for ingress of fluid within the bioreactor vessel,
wherein the harvest tube may be advanced to an extended position with the terminal end thereof within an interior of the bioreactor vessel such that fluid within the bioreactor vessel may be extracted through the one or more holes and into the harvest tube.

2. The valve assembly of claim 1, further including a gate valve within the harvest tube that alternately occludes and permits flow through the one or more holes in a side wall of the harvest tube.

3. The valve assembly of claim 2, wherein the gate valve includes a rotating member sealed for rotation within the harvest tube having one or more apertures that may be aligned with the one or more holes in the harvest tube to permit flow through the one or more holes in the harvest tube.

4. The valve assembly of claim 3, wherein the rotating member connects via a slender rod passing through the harvest tube to a lever for manual rotation thereof.

5. The valve assembly of claim 1, wherein the harvest tube has an outer end secured within a mounting block and open to a bore in the mounting block, the assembly further including a fluid connector open to the bore and adapted to couple to tubing for removal of fluid from within the bioreactor vessel.

6. The valve assembly of claim 5, further including a flexible sheath attached to the mounting block and extending around the harvest tube to the harvest port, the sheath being extended when the harvest tube is in its retracted position and collapsed when the harvest tube is in its extended position.

7. The valve assembly of claim 6, wherein the flexible sheath has at least one vent to alleviate positive or negative pressure generation between the sheath and harvest tube during harvest tube movement.

8. The valve assembly of claim 5, further including a locking assembly adapted to be fixed relative to the harvest port and secured to the mounting block for locking the position of the harvest tube relative to the harvest port.

9. The valve assembly of claim 1, wherein the bioreactor vessel is a disposable bag with flexible walls and the harvest port is heat sealed to one of the flexible walls.

10. The valve assembly of claim 9, wherein the harvest port is heat sealed to a lower curved flexible wall of the disposable bag such that the harvest tube extends upward into an interior of the bag from the retracted to the extended position.

11. A bioreactor harvest valve assembly, comprising:
a. a harvest port adapted to be mounted in and through a wall of a bioreactor vessel and having a throughbore;
b. a harvest tube configured to pass through the throughbore of the harvest port which has seals therein to prevent leakage around the harvest tube, the harvest tube having a cap at a terminal end that seals against the harvest port and closes the throughbore in a retracted position of the harvest tube;
c. a gate valve within the harvest tube that alternately occludes and permits flow through the harvest tube;
d. tubing in fluid communication with the harvest tube,
wherein the harvest tube may be advanced to an extended position with the terminal end thereof within an interior of the bioreactor vessel such that fluid within the bioreactor vessel may be extracted through the gate valve into the harvest tube and into the tubing.

12. The valve assembly of claim 11, wherein the harvest tube has one or more holes formed in a side wall thereof, and the gate valve alternately occludes and permits flow through the one or more holes.

13. The valve assembly of claim 12, wherein the gate valve includes a rotating member sealed for rotation within the harvest tube having one or more apertures that may be aligned with the one or more holes in the harvest tube to permit flow through the one or more holes in the harvest tube.

14. The valve assembly of claim 13, wherein the rotating member connects via a slender rod passing through the harvest tube to a lever for manual rotation thereof.

15. The valve assembly of claim 11, wherein the harvest tube has an outer end secured within a mounting block and open to a bore in the mounting block, the assembly further including a fluid connector open to the bore and adapted to couple to tubing for removal of fluid from within the bioreactor vessel.

16. The valve assembly of claim 15, further including a flexible sheath attached to the mounting block and extending around the harvest tube to the harvest port, the sheath being extended when the harvest tube is in its retracted position and collapsed when the harvest tube is in its extended position.

17. The valve assembly of claim 16, wherein the flexible sheath has at least one vent to alleviate positive or negative pressure generation between the sheath and harvest tube during harvest tube movement.

18. The valve assembly of claim 15, further including a locking assembly adapted to be fixed relative to the harvest port and secured to the mounting block for locking the position of the harvest tube relative to the harvest port.

19. The valve assembly of claim 11, wherein the bioreactor vessel is a disposable bag with flexible walls and the harvest port is heat sealed to one of the flexible walls.

20. The valve assembly of claim 19, wherein the harvest port is heat sealed to a lower curved flexible wall of the disposable bag such that the harvest tube extends upward into an interior of the bag from the retracted to the extended position.
